(19) United States
(12) Patent Application Publication
(10) Pub. No.: US 2023/0255817 A1
(43) Pub. Date: Aug. 17, 2023

(54) Ring that locks to a condoms elastic band

(71) Applicant: Kevin Jackson, Suffield, CT (US)

(72) Inventor: Kevin Jackson, Suffield, CT (US)

(21) Appl. No.: 18/445,053

(22) Filed: Mar. 21, 2023

Related U.S. Application Data
(60) Provisional application No. 63/372,644, filed on Mar. 28, 2022.

(51) Int. Cl. A61F 6/04

(52) U.S. Cl. CPC A61F 6/04; A61F 2006/047

(57) ABSTRACT

A ring that locks to a condoms elastic band that is put on the penis before the condom so the condom can have its elastic band pulled into a groove along the center of the ring. It has two sides that slide open and shut over the top of the condoms elastic band to lock it in place on the ring. The ring is shaped wider then the hole of the vagina so it hooks onto the outer area of the vagina so that the condom cannot slide off during sex even if there is lubrication inside the condom.

### FIELD

The present application related to a washer shaped ring that attaches to a condom with two parts on both sides that push inwardly over the condoms elastic band to lock onto the band.

### BACKGROUND

Condoms are used to lower the risk of sexually transmitted diseases and for birth control but they have the tendency to rupture or slide off during sexual encounters and they can slide into the vagina. Also they cause a blockage of direct contact of the genitals which results in less pleasure for both the male and female. If there is any lubricant applied to the inside of the condom then the slippage problem is almost certain.

On solution to the slippage problem similar to the present application is presented by Van Der Valk (U.S. Pat. No. 5,163,449) who invented a similar ring that attaches to a condoms back end but is assembled by the user in a different manner and is less effective. Van Der Valk's ring has two different forms. The first one has a basic ring that is pushed onto the penis as a ring fits on a finger. Then it has a larger peripheral groove on its outer circumference and a circular groove on its inner circumference near the hollowed hole in its middle where the condom is slid on. The condoms elastic band at its back end is stretched over the inner groove after the condom is rolled on and then another ring is slide on which has a circular outer perimeter then the surface of the penis which prevents the condom from slipping off according to Van Der Valk. The problem with this version is that there is nothing holding the condom on the ring except the fact that its band is stretched over a groove because its second ring is not designed to cover the band but only to rest on the outer part of the basic ring. If there is any extra room on the penis behind the condoms length and the male pushed with any slight force the condom would easily push off the ring. Also it requires two rings making the process very difficult.

Van Der Valk's second version is a condom that is made with a ridgid and wider back end that is slid onto its basic ring which has a perimeter that rises significantly wider then the perimeter of the surface of the condom and has a second snap ring that is slid on and snaps into the basic ring. Thereby it sandwiches the condoms ridigid circular back end, between the rings. The problem with this version is that the back end of the condom has to be made with a rigid material which prevents it from being rolled for packaging. When putting a condom on a male it is important to be rolled on to prevent air bubbles from entering inside which can cause ruptures. A second major problem with it is that it would not allow a user to choose any condom brand with a common style and it also does not allow the ring to be used more then once. Given the circumstances, an improved ring with a more simple connection that is compatible with any condom and a more secure mechanism all in one device presents itself.

### SUMMARY

By providing a condom ring that locks the condoms elastic band into it the person can add as much lubricate as he desires inside the condom and still use any brand of condom he prefers. Also he can easily put the ring on in the dark and not have to attach multiple rings with frivolous attachment.

The disclosed embodiments provide a ring with two side parts that push inwardly and locks the condoms elastic band inside of them.

In an embodiment the ring has a circular groove which the condoms elastic band stretches around and is locked in by two sliding sides that push inwardly over the band locking it to the ring, and the ring has an abrupt upward ninety degree turn at least rising one centimeter, perpendicular from the surface of the hollow circumference in its middle and is circular.

Further objects, features, advantages and properties of the device according to the present application will become apparent from the detailed description.

### BRIEF DESCRIPTION

In the following detailed description of the present application teachings of the present application will be explained in more detail with references to the example embodiments shown in the drawings, in which:

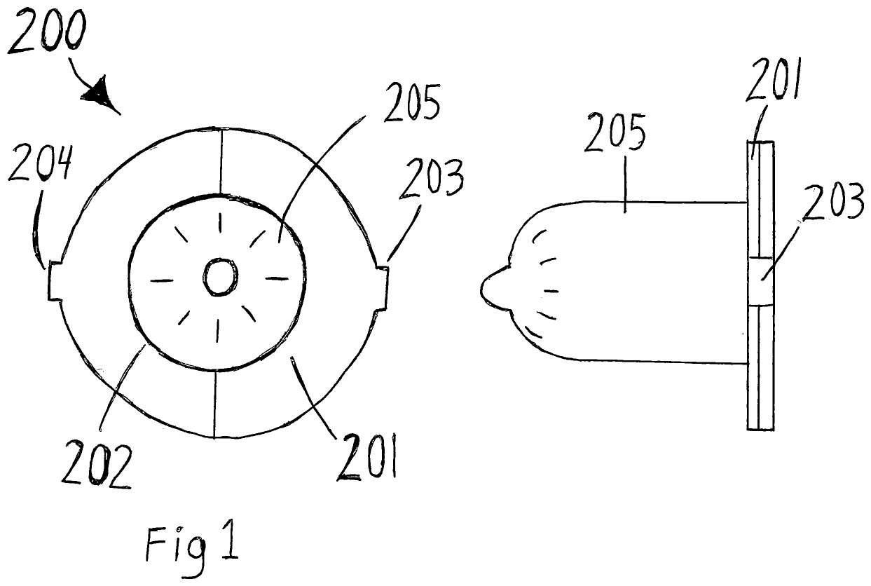

FIG. 1 is an isometrical view of the condom ring with a condom locked inside of the two sliding locks which are closed shut.

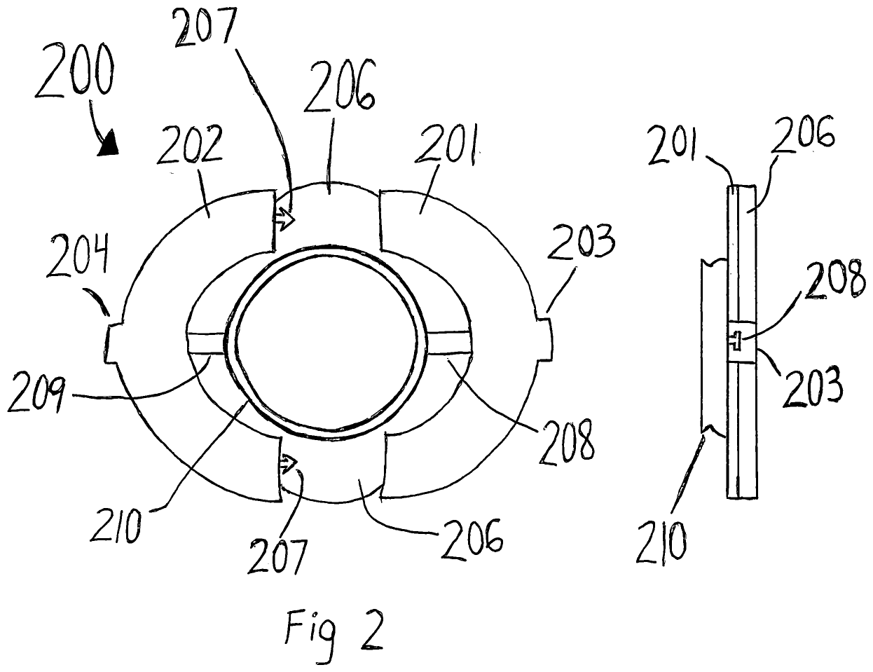

FIG. 2 is an isometrical view of the condom ring but without a condom on it and the sliding locks are open.

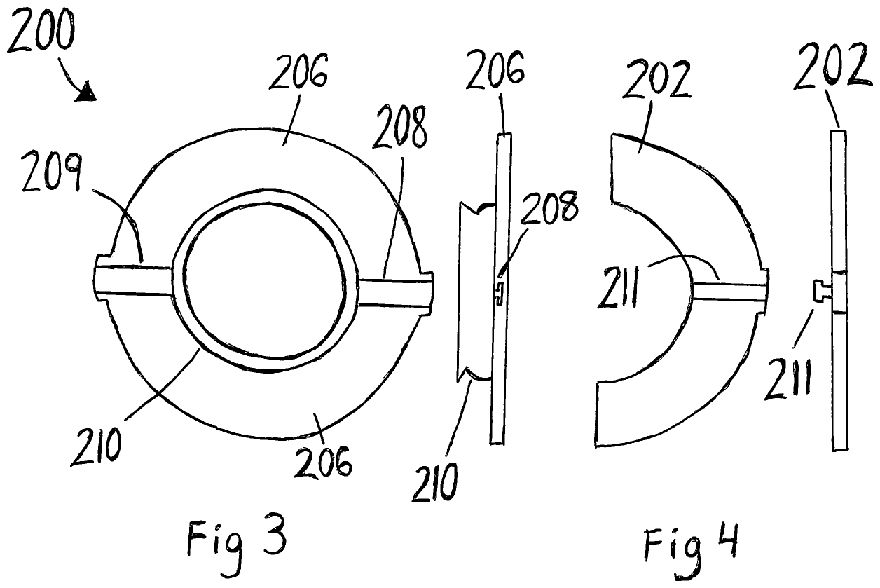

FIG. 3 is an isometrical view of the condom ring without the condom and without the two sliding locks attached.

FIG. 4 is a view of the backwards side of one of the sliding side locks without the ring that it attaches to.

### DETAILED DESCRIPTION

In the following detailed description, the device according to the teachings for this application in the form of a condom ring will be described by the embodiments.

A first embodiment of the condom ring 200 is illustrated in FIG. 1. The condom ring 200 has two halfs of a circular ring 202 and 201 connected to a condom 205. The condom 205 is unrolled and is facing forward so only the tip, or closed end is shown. The ring 202 has a hollow circular inside which is where the opened end of the condom 205 is first stretched around a circular groove 210 which will be later shown in FIG. 3. There are two sides of the ring 200, a left side 202 and a right side 201 which both slide outward to open and slide inward to lock and clamp the condom 205 in by its band into the groove 210. There is a left side grip 204 and a right side grip 203 which the user grabs to slide the condom ring 200 open and closed. The right side 201 and left side 202 are an inch in width from above its middle circular opening so it can catch onto the outer area of a womens vagina and prevent the condom 205 from being able to slip off into the vagina regardless of how lubricated the inside is. The condom ring 200 is flat as the shape of a washer is which is used with a screw and does not take up any space on or above the condom except for on and above its very back end and does not enter inside the vagina.

A second embodiment of the condom, ring 200 is illustrated in FIG. 2. The circular ring 200 is now shown with its left side 202 extended open and its right side 201 extended open. The sides 201 and 202 are made with soft rubber on the outer area because that is the part that touches around the vagina. When the right side 201 and the left side 202 are slid open you can see that there is a back side ring 206 which is the same shape of the circular sides 201 and 202 when they are closed. The only part of the two front sides 201 and 202 that protrudes further then the back side ring 206 is the two grips 203 and 204 on the sides of the front 201 and 202. The back side ring 206 has a left side rail 209 and a right side rail 208 that has an inner groove that form a straight line through the center across which hooks later explained in FIG. 4 fit inside in order to simply slide the two sides 201 and 202 open and closed. On the edges of the left side 202 where it meets the right side 201 there are hooks 207 which snap into the right side 201 to keep them closed. There is a center ring 210 which rises perpendicular from the surface of the back side ring 206 and it has a groove along its outer side which is where the condoms band fits inside which would be slightly wider then the circumference of a common condoms band so it remains in place when it is stretched around it into the groove. When the left side 202 and the right side 201 close shut, they fit snuggly over the condoms band locking it onto the center ring 210.

A Third embodiment of the condom ring 200 is illustrated in FIG. 3. The condom ring 200 is shown without the left side 202 or the right side 201 on. The inner ring 206 is a full circle and has the left side rail 209 and the right side rail 208 formed directly across the center and has an inner hole along the inside of both sides which hooks previously described on the reverse sides of the two sliding sides 201 and 202 fit into. The hooks cannot be seen and will be described and shown in FIG. 4.

A fourth embodiment of the condom ring 200 is illustrated in FIG. 4. The left side of the sliding sides is shown alone. There is a hook 211 that rises up along the center which hooks into the inner rail 209 on the reverse side of the left side 202 previously described and allows it to move side to side. The right side 201 would obviously have the same hook 211 as the left side has.

The teaching of the present application has numerous advantages. Different embodiments or implementations may yield one or more advantages. It should be noted that this is not an exhaustive list and there may be other advantages which are not described herein.

Although the teaching of the present application has been described in detail for purpose of illustration it is understood that such detail is solely for that purpose and variations can be made therein by those skilled in the art without departing from the scope of the teachings.

Features described in the preceding description may be used in combinations other then the combinations explicitly described.

Whilst endeavoring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

The term “comprising” as used in the claims does not exclude other elements or steps. The term “a” or “an” as used in the claims does not exclude a plurality. A unit or other means may fulfill the functions of several units or means recited in the claims.

1. A ring that locks to a condoms elastic band, comprising: a flat back end ring that is the shape of a washer used with a screw and has a hollow center hole that has a circumference that is slightly larger then the circumference of the elastic band on the average sized condom and is at least one centimeter wide from its middle hollow circumference, and has an inner track or rail straight across its middle from side to side which a hook can go inside of; a center ring that is not flat but is the shape of a ring that fits on a finger and is connected to the said back end ring at its hollow center rising perpendicular away from the surface of the said back end ring and has an inner groove along its outer side that the band of a condom can fit into; and a front ring identical in circumference to the said back end ring that splits in two halves on each side and has a hook on the reverse side of each side which fits into the inner track or rail of the said back end ring so that each half of the front ring can slide open and closed. The front rings two side close to fit snugly over top of the said center rings outer grooves so that it locks the condoms elastic band to the said center rings outer groove. There are two hooks on the front rings left sides edge where it meets the edge of the right side which locks inside a hole in the edge of the right side so that it locks the two sides closed.
